(19) United States
(12) Patent Application Publication
(10) Pub. No.: US 2013/0296691 A1
(43) Pub. Date: Nov. 7, 2013

(54) MAGNETICALLY TRACKED SURGICAL NEEDLE ASSEMBLY

(71) Applicant: WESTLEY ASHE, MILTON, VT (US)

(72) Inventor: WESTLEY ASHE, MILTON, VT (US)

(73) Assignee: ASCENSION TECHNOLOGY CORPORATION, MILTON, VT (US)

(21) Appl. No.: 13/464,238

(22) Filed: May 4, 2012

(51) Int. Cl. A61B 5/05

(52) U.S. Cl. 600/424

(57) ABSTRACT

An electromagnetic needle tracking system includes a needle assembly, a calibration system and a computing system. The needle assembly includes a needle stylet and a sensor assembly. The sensor assembly includes a sensor that measures position and angular orientation data when placed within an electromagnetic field. The calibration system measures the sensor's position and angular orientation for a known needle tip position and angular orientation within a calibration fixture and calculates a position offset and an angular orientation offset of the sensor relative to the needle tip position and angular orientation. The computing system computes position and angular orientation data of the needle tip by adding the sensor position offset and angular orientation offset to the measured position and angular orientation data, respectively.

### FIELD OF THE INVENTION

The present invention relates to a magnetically tracked needle assembly, and more particularly to a disposable, magnetically tracked needle assembly, that includes a sensor coaxially inserted into a disposable stylet.

### BACKGROUND OF THE INVENTION

Magnetic tracking of instruments with respect to imaged anatomy is widely employed in medical practice. Imaging systems that are enhanced with magnetic tracking may be used to track and display position and orientation of a biopsy needle assembly relative to the imaging plane. They help the clinician guide the instrument to a chosen target with reduced error compared to an unguided biopsy needle. Furthermore, the visual representation of the tracked biopsy needle is not constrained to the ultrasound imaging plane, thus enabling the clinician with more freedom of motion.

For magnetic tracking of an instrument, an electromagnetic sensor is included in a location of the instrument. Electromagnetic sensors are usually electromagnetic coils that surround or are close to the objects whose location is being tracked. When the instruments with the included sensors are placed within a varying electromagnetic field a voltage is generated in the electromagnetic sensor. This generated voltage is used to determine and track the locations and relative positioning of the instrument, within the electromagnetic field. Ultrasound system enhanced with magnetic tracking of sensors, displays 3-dimensional merger of ultrasound generated anatomical features and the visual representation of the instrument position and orientation.

Prior art magnetic tracking systems require that the sensor be located in a predetermined location inside the instrument. This is accomplished by precisely controlling the physical location of the sensor within a tube containing the instrument. Such precision increases the complexity and the time required to manufacture the sensor assembly. These prior art tracking system usually do not provide a way to correct small manufacturing errors which are outside of the allowed positioning tolerances. Prior art tracking systems also do not provide a way for correcting the angular misalignment caused by tolerances in the sliding fit between the sensor assembly and the elongated instrument. This orientation error is generally greater than that inherent in the magnetic tracking system and results in the system displaying an incorrect trajectory for the instrument.

Medical procedures are increasingly cost driven. Magnetic tracking, while improving procedure outcomes, adds a cost element to the equipment. Labor cost associated with assembly and test of needle increases with design and process complexity. The needle sensor position and orientation relative to the stylet tip position and orientation is unique, with calibration data associated with each needle assembly produced. Overhead costs associated with serial number traceability maintenance of this needle assembly sensor calibration data are hidden, but significant.

It is desirable to have an electromagnetically tracked needle that is cost effective, provides accurate needle position and angular orientation and does not require repeated sterilization.

### SUMMARY OF THE INVENTION

This invention relates to electromagnetic tracking of medical instruments, specifically tracking of minimally invasive instruments such as biopsy needles, ablation instruments, and various other instruments for which the end of a cannula must be precisely placed to enable diagnostic or interventional procedures at the distal end of the cannula. Such procedures are often performed using an imaging modality such as ultrasound, where electromagnetic tracking of the instrument allows spatial location and visualization of anatomy and instrument location.

In general, in one aspect, the invention features an electromagnetic needle tracking system including a needle assembly, a calibration system and a computing system. The needle assembly includes a needle stylet and a sensor assembly. The needle stylet includes an elongated hollow tube having an open proximal end and a distal end comprising a needle tip. The sensor assembly includes an elongated body and a sensor attached to the elongated body and the elongated body is shaped and dimensioned to be inserted into the elongated hollow tube. The sensor is configured to measure position and angular orientation data when placed within an electromagnetic field. The calibration system includes a calibration fixture and is configured to measure the sensor's position and angular orientation for a known needle tip position and angular orientation within the calibration fixture and to calculate a position offset and an angular orientation offset of the sensor relative to the needle tip position and angular orientation. The computing system computes position and angular orientation data of the needle tip by adding the sensor position offset and angular orientation offset to the measured position and angular orientation data, respectively.

Implementations of this aspect of the invention may include one or more of the following features. The system may further include a non-volatile storage circuitry configured to store the calculated sensor position and angular orientation offsets. The needle stylet further includes a stylet receiver attached to the proximal end of the elongated hollow tube and the needle assembly further includes means for attaching the elongated body's proximal end to the stylet receiver. The elongated body's proximal end is attached to the stylet receiver with an adhesive. The adhesive may be cyanoacrylate, epoxy, hot melt or solvent bonding. The stylet receiver includes a cavity and the cavity is tapered. The sensor assembly further includes an insulated cable and a pair of twisted insulated wires connected to the distal end of the insulated cable. The distal end of the insulated cable is inserted in the receiver cavity and the proximal end is connected to the computing system. The tapered cavity provides a hard stop for the inserted distal end of the insulated cable. The stylet receiver includes a cavity extending coaxially with the elongated hollow tube. The stylet receiver includes a cavity extending parallel to but offset from the elongated hollow tube. The assembly may further include an outer cannula and the needle stylet is configured to be inserted into the outer cannula.

In general, in another aspect, the invention features a needle assembly including a needle stylet, a sensor assembly and a computing system. The needle stylet includes an elongated hollow tube and a needle. The elongated hollow tube extends along a first axis and has an open proximal end and a closed distal end. The needle is attached to the closed distal end of the elongated hollow tube and has a tip end that extends a first distance from the closed distal end of the elongated hollow tube along the first axis. The sensor assembly includes an elongated body extending along a second axis, and a sensor placed at a second distance from the distal end of the elongated body. The elongated body is shaped and dimensioned to be inserted into the elongated hollow tube so that the distal end of the elongated body is placed in contact with the closed distal end of the elongated hollow tube. The sensor is configured to measure position and angular orientation data when placed within an electromagnetic field. The computing system computes the position and angular orientation of the tip end of the needle by adding the sum of the first and the second distances to the measured position data and by adding the angular difference between the first and second axes to the measured angular orientation data, respectively. The assembly may further include a non-volatile storage circuitry configured to store calibration data comprising the first and second distances, the sum of the first and second distances, and the angular difference between the first and second axes.

In general, in another aspect, the invention features a needle assembly including a needle stylet, a sensor assembly and a computing system. The needle stylet includes an elongated hollow tube and a needle. The elongated hollow tube extends along a first axis and has an open proximal end and a closed distal end. The needle is attached to the closed distal end of the elongated hollow tube and has a tip end that extends a first distance from the closed distal end of the elongated hollow tube along the first axis. The sensor assembly includes an elongated body extending along a second axis, and a sensor located at a second distance from the distal end of the elongated body. The elongated body is shaped and dimensioned to be inserted into the elongated hollow tube so that the distal end of the elongated body is placed in contact with the closed distal end of the elongated hollow tube. The sensor is configured to measure position and angular orientation data when placed within an electromagnetic field. The system also includes means for fixing the sensor's angular orientation within the elongated hollow tube to be coaxial with the first axis. The computing system computes the position of the tip end of the needle by adding the sum of the first and the second distances to the measured position data.

Implementations of this aspect of the invention may include one or more of the following features. The means for fixing the sensor's angular orientation includes a sleeve of hot-melt plastic and the sleeve is configured to be positioned around the elongated body and to be tacked to the elongated body by heating. The sensor's angular orientation is fixed to be coaxial with the first axis by iteratively heating and melting the sleeve of hot-melt plastic, orienting the elongated body, cooling and solidifying the sleeve of hot-melt plastic and measuring the resulting angular difference between the first and second axes until the elongated body is coaxial with the elongated hollow tube. The sensor may be a magnetic sensor and the elongated body is oriented within the elongated hollow tube by applying a magnetic force. The means for fixing the sensor's angular orientation include first and second heat-shrink rings. The first and second heat-shrink rings are positioned coaxially and around the sensor's first and second ends, respectively, and subsequently the sensor assembly is inserted into the elongated hollow tube and the heat-shrink rings are heated at a controlled temperature and for a controlled time period until the outer diameter of the heat-shrink rings expands to be slightly smaller than the inner diameter of the elongated hollow tube, and thereby orienting and fixing the elongated body coaxially with the elongate hollow tube. The outer surface of each of the first and second heat-shrink rings has a groove and the groove is oriented parallel to the ring's axis.

In general, in another aspect, the invention features a needle assembly including a needle stylet, a sensor assembly and a computing system. The needle stylet includes an elongated hollow tube and a needle. The elongated hollow tube extends along a first axis and has an open proximal end and a closed distal end. The needle is attached to the closed distal end of the elongated hollow tube and has a tip end that extends a first distance from the closed distal end of the elongated hollow tube along the first axis. The sensor assembly includes an elongated body, a sensor located at the distal end of the elongated body and a stop-plug configured to be placed over the elongated body's distal end. The elongated body is shaped and dimensioned to be inserted into the elongated hollow tube so that the distal end of the stop-plug is in contact with the closed distal end of the elongated hollow tube. The stop-plug has an outer diameter slightly smaller than the inner diameter of the elongated hollow tube and is configured to orient the elongated body coaxially with the elongated hollow tube. The stop-plug has an inner diameter slightly larger than the outer diameter of the elongated body and is configured to receive and place the elongated body's distal end at a second distance from the stop-plug's distal end. The computing system computes the position of the tip end of the needle by adding the sum of the first and the second distances to the measured position data.

Implementations of this aspect of the invention may include one or more of the following features. The outer surface of the stop-plug comprises a groove and the groove is oriented parallel to the stop-plug's axis. The needle stylet further includes a stylet receiver attached to the proximal end of the elongated hollow tube and the needle assembly further includes means for attaching the elongated body's proximal end to the stylet receiver.

In general, in another aspect, the invention features method for electromagnetic tracking of a needle including providing a needle assembly, providing a calibration system and providing a computing system. The needle assembly includes a needle stylet and a sensor assembly. The needle stylet includes an elongated hollow tube having an open proximal end and a distal end comprising a needle tip. The sensor assembly includes an elongated body, and a sensor attached to the elongated body. The elongated body is shaped and dimensioned to be inserted into the elongated hollow tube, and the sensor is configured to measure position and angular orientation data when placed within an electromagnetic field. The calibration system includes a calibration fixture and is configured to measure the sensor's position and angular orientation for a known needle tip position and angular orientation within the calibration fixture and to calculate a position offset and an angular orientation offset of the sensor relative to the needle tip position and angular orientation. The computing system computes position and angular orientation data of the needle tip by adding the sensor position offset and angular orientation offset to the measured position and angular orientation data, respectively.

In general, in another aspect, the invention features a method for electromagnetic tracking of a needle including providing a needle stylet, providing a sensor assembly and providing a computing system. The needle stylet includes an elongated hollow tube and a needle and the elongated hollow tube extends along a first axis and has an open proximal end and a closed distal end. The needle is attached to the closed distal end of the elongated hollow tube and has a tip end that extends a first distance from the closed distal end of the elongated hollow tube along the first axis. The sensor assembly includes an elongated body extending along a second axis, and a sensor placed at a second distance from the distal end of the elongated body. The elongated body is shaped and dimensioned to be inserted into the elongated hollow tube so that the distal end of the elongated body is placed in contact with the closed distal end of the elongated hollow tube. The sensor is configured to measure position and angular orientation data when placed within an electromagnetic field. The computing system computes the position and angular orientation of the tip end of the needle by adding the sum of the first and the second distances to the measured position data and by adding the angular difference between the first and second axes to the measured angular orientation data, respectively.

In general, in another aspect, the invention features a method for electromagnetic tracking of a needle including providing a needle stylet, providing a sensor assembly and providing a computing system. The needle stylet includes an elongated hollow tube and a needle. The elongated hollow tube extends along a first axis and has an open proximal end and a closed distal end. The needle is attached to the closed distal end of the elongated hollow tube and has a tip end that extends a first distance from the closed distal end of the elongated hollow tube along the first axis. The sensor assembly includes an elongated body extending along a second axis, and a sensor located at a second distance from the distal end of the elongated body. The elongated body is shaped and dimensioned to be inserted into the elongated hollow tube so that the distal end of the elongated body is placed in contact with the closed distal end of the elongated hollow tube. The sensor is configured to measure position and angular orientation data when placed within an electromagnetic field. The computing system computes the position of the tip end of the needle by adding the sum of the first and the second distances to the measured position data. The method also includes fixing the sensor's angular orientation within the elongated hollow tube to be coaxial with the first axis. The sensor's angular orientation may be fixed via a sleeve of hot-melt plastic and the sleeve is configured to be positioned around the elongated body and to be tacked to the elongated body by heating. The sensor's angular orientation may be fixed via first and second heat-shrink rings. The first and second heat-shrink rings are positioned coaxially and around the sensor's first and second ends, respectively, and subsequently the sensor assembly is inserted into the elongated hollow tube and the heat-shrink rings are heated at a controlled temperature and for a controlled time period until the outer diameter of the heat-shrink rings expands to be slightly smaller than the inner diameter of the elongated hollow tube, and thereby orienting and fixing the elongated body coaxially with the elongate hollow tube.

In general, in another aspect, the invention features a method for electromagnetic tracking of a needle including providing a needle stylet, providing a sensor assembly and providing a computing system. The needle stylet includes an elongated hollow tube and a needle. The elongated hollow tube extends along a first axis and has an open proximal end and a closed distal end. The needle is attached to the closed distal end of the elongated hollow tube and has a tip end that extends a first distance from the closed distal end of the elongated hollow tube along the first axis. The sensor assembly includes an elongated body, a sensor located at the distal end of the elongated body and a stop-plug configured to be placed over the elongated body's distal end. The elongated body is shaped and dimensioned to be inserted into the elongated hollow tube so that distal end of the stop-plug is in contact with the closed distal end of the elongated hollow tube. The stop-plug has an outer diameter slightly smaller than the inner diameter of the elongated hollow tube and is configured to orient the elongated body coaxially with the elongated hollow tube. The stop-plug has an inner diameter slightly larger than the outer diameter of the elongated body and is configured to receive and place the elongated body's distal end at a second distance from the stop-plug's distal end. The computing system computes the position of the tip end of the needle by adding the sum of the first and the second distances to the measured position data.

Among the advantages of this invention may be one or more of the following. The invention provides a cost-reduced sensor assembly, where costs associated with assembly labor, traceability overhead, high inventory mix, and component materials are minimized. The low cost of the sensor assembly enables a single-use protocol, thus eliminating the procedural cost, borne by the end-user, of repeated sterilization for re-uses. The invention also provides a single-use disposable needle configuration, that eliminates the substantial cost of repeatedly sterilizing the sensor assembly. The invention also provides means for correcting the angular misalignment caused by tolerances in the sliding fit between the sensor assembly and the elongated instrument, or stylet axis.

The details of one or more embodiments of the invention are set forth in the accompanying drawings and description below. Other features, objects, and advantages of the invention will be apparent from the following description of the preferred embodiments, the drawings, and the claims.

### BRIEF DESCRIPTION OF THE DRAWINGS

Referring to the figures, wherein like numerals represent like parts throughout the several views:

FIG. 1 illustrates an ultrasound imaging system enhanced with magnetic instrument tracking;

FIG. 2A is an exploded perspective diagram of a magnetically tracked, disposable needle assembly, according to this invention;

FIG. 2B and FIG. 2C are magnified views of two different embodiments of the stylet receiver (area A of FIG. 2A), according to this invention;

FIG. 2D-FIG. 2G are magnified views of four different sensor configuration embodiments (area B of FIG. 2A), according to this invention;

FIG. 3A-FIG. 3C are schematic views of the components and methods for measuring, positioning and orientating the electromagnetic sensor of FIG. 2D relative to the stylet tip of FIG. 2A;

FIG. 4A-FIG. 4D are schematic views of the components and methods for measuring, positioning and orientating the electromagnetic sensor of FIG. 2E in a stylet tip of FIG. 2A;

FIG. 5A-FIG. 5D are schematic views of the components and methods for measuring, positioning and orientating the electromagnetic sensor of FIG. 2F in a stylet tip of FIG. 2A;

FIG. 6A-6D are schematic views of the components and methods for measuring, positioning and orientating the electromagnetic sensor of FIG. 2G in a stylet tip of FIG. 2A;

FIG. 7A is a schematic perspective view of the press-fit capture of the sensor cable in the coaxial stylet receiver cavity of FIG. 2B;

FIG. 7B is a magnified view of area C in FIG. 7A;

FIG. 7C is a magnified view of area D in FIG. 7A;

FIG. 8A is a schematic perspective view of the press-fit capture of the sensor cable and fixing of the sensor tube in an axially offset receiver cavity of FIG. 2C;

FIG. 8B is a magnified view of area E in FIG. 8B;

FIG. 9 is a schematic perspective view of the fully assembled disposable needle, according to this invention.

### DETAILED DESCRIPTION OF THE INVENTION

In a biopsy application, precise knowledge of needle tip position and orientation is critical. In such applications, it is optimal to locate the magnetic sensor as close to the distal end of the needle as is practical, because the needle flexes while penetrating anatomy. Furthermore, care must be taken to ensure that pathogens are not passed from patient to patient using this assembly, thus component sterilization and reprocessing are required if the sensor assembly is to be re-used. A single-use disposable configuration, eliminates the substantial cost to repeatedly sterilize the sensor-assembly. The invention described herein is motivated by the need for a cost-reduced sensor assembly, where costs associated with assembly labor, traceability overhead, high inventory mix, and component materials are minimized. The low cost of the sensor assembly enables a single-use protocol, thus eliminating the procedural cost, borne by the end-user, of repeated sterilization for re-uses.

The present invention addresses the problem of positioning and orientating the magnetic sensor relative to a stylet tip. Furthermore, the present invention addresses the problem of fastening the sensor cable and sensor tube proximal end into a stylet receiver.

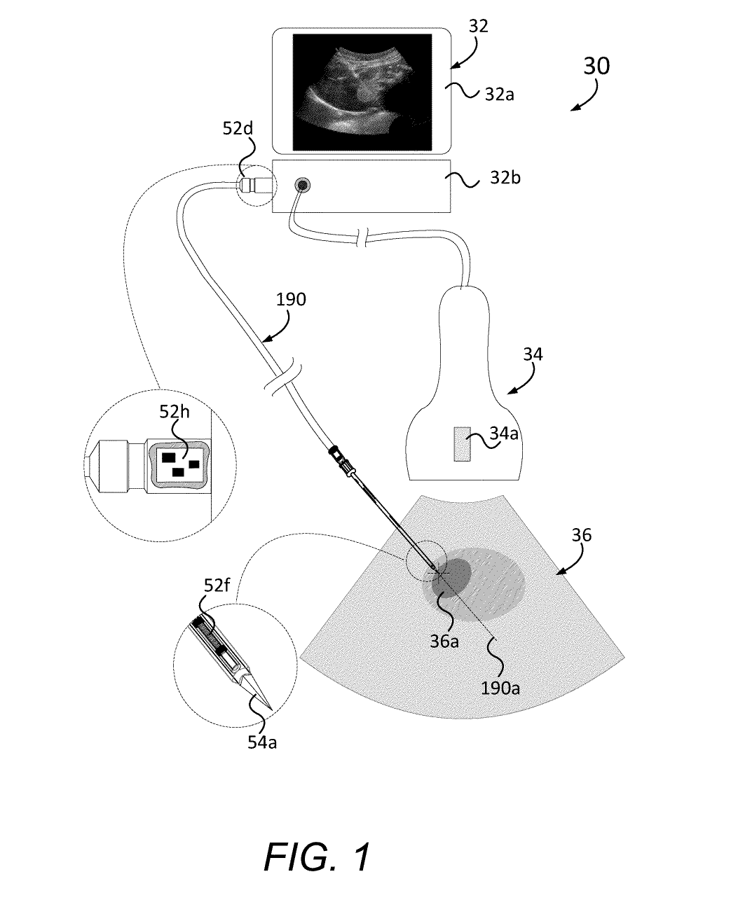

Referring to FIG. 1, imaging tools, such as ultrasound system 30, are used to image detailed anatomical features in a spatial slice (or imaging plane) 36. Ultrasound system 30 includes a hand-held probe 34, a display 32a and electronics 32b. For magnetic tracking of an instrument 190 with ultrasound system 30, electromagnetic sensors 34a and 52f are included in the hand-held ultrasound probe 34 and in a location of instrument 190, respectively. Sensors 34a and 52f are usually electromagnetic coils that surround or are close to the objects whose location is being tracked. In the example of FIG. 1, instrument 190 is a needle assembly and sensor 52f is close to the needle tip 190a. When sensor 52f is placed within a varying electromagnetic field a voltage is generated in the electromagnetic sensor 52f. Similarly, when hand-held ultrasound probe 34 with the embedded sensor 34a is placed within the varying electromagnetic field a voltage is generated in the electromagnetic sensor 34a. These generated voltages in sensors 34a, 52f are used to determine and track the locations and relative positioning of ultrasound probe 34 and needle tip 190a, respectively, within the electromagnetic field. Ultrasound system 30 enhanced with magnetic tracking of sensors 34a 52f, displays the 3-dimensional merger of ultrasound generated anatomical features 36a and the visual representation of the instrument position and orientation 190a.

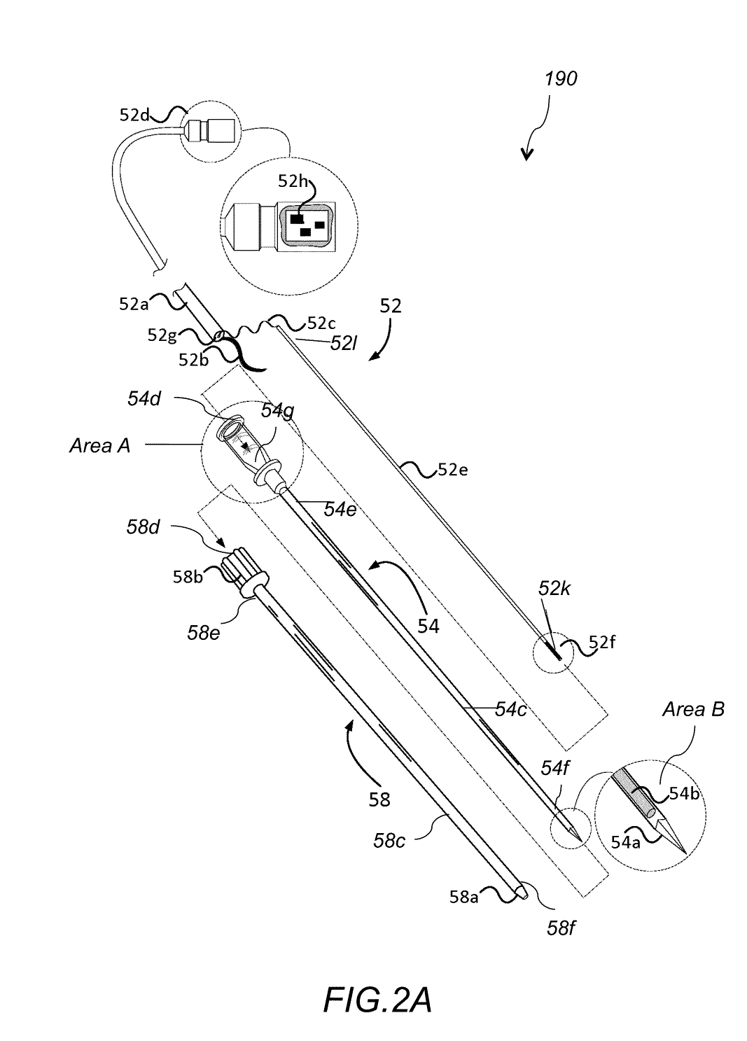

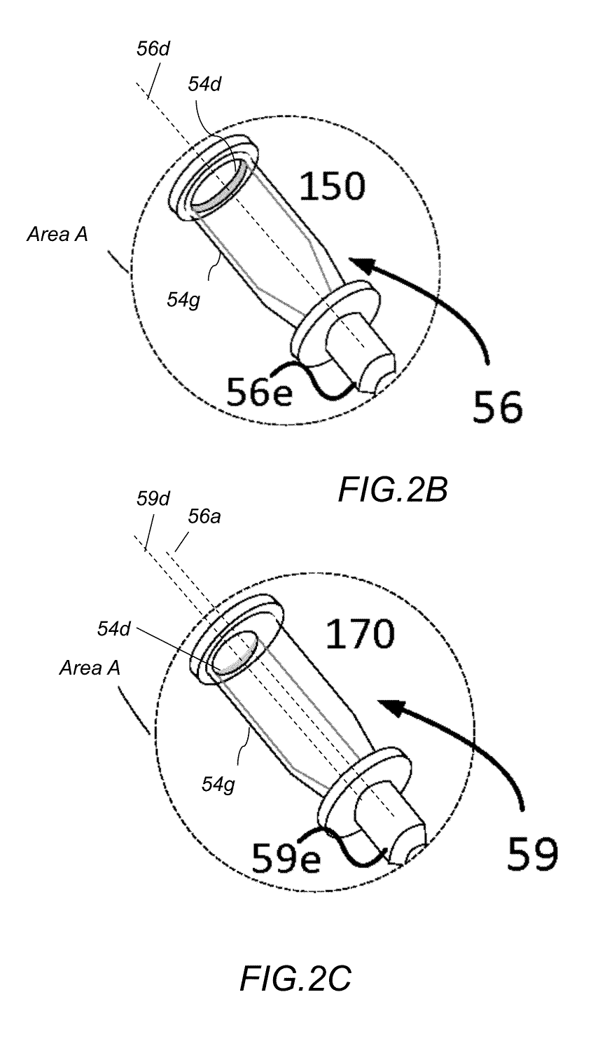

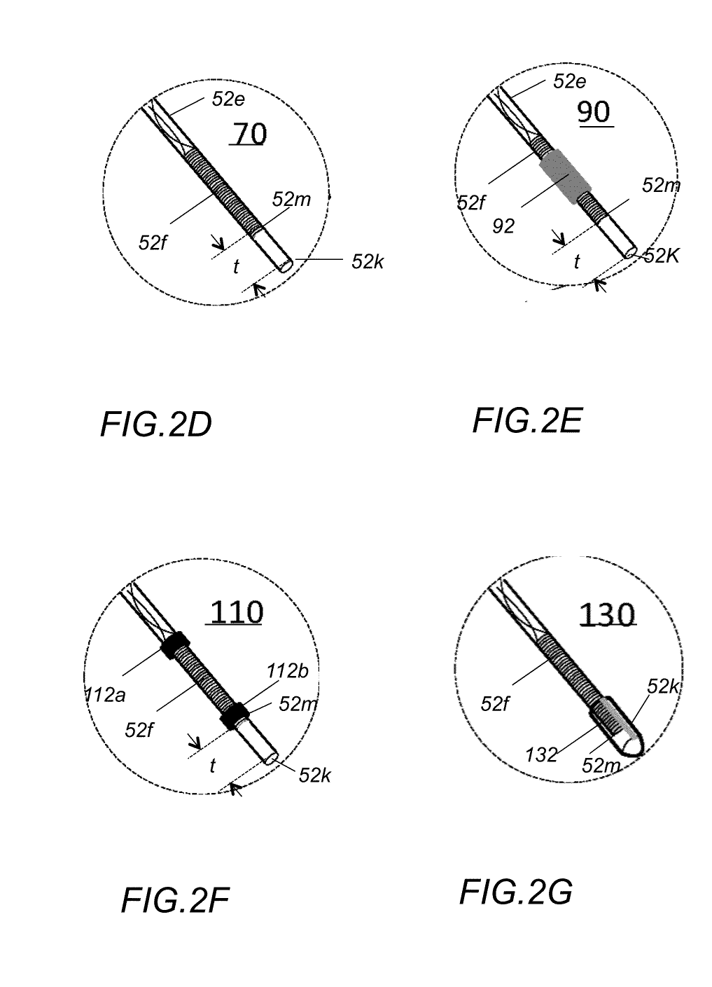

Referring to FIG. 2A, needle assembly 190 includes an outer cannula 58, a stylet 54, and an electromagnetic sensor assembly 52. Outer cannula 58 includes an elongated tube 58c having a plastic receiver 58b at the proximal end 58e and a cannula tip 58a at the distal end 58f. Outer cannula 58 also includes an axial though-opening 58d extending the entire length of the elongated tube 58c. Through-opening 58d is shaped and dimensioned to match and receive the stylet 54. Cannula tip 58a is often a spade or a truncated cone, as shown in FIG. 2A. The truncated cone tip form 58a is common for soft-tissue biopsy applications and is dimensioned to follow the stylet tip 54a during tissue insertion.

Stylet 54 includes an elongated hollow tube 54c having a stylet receiver/handle 54g at the proximal end 54e and a sharp tip 54a at the distal end 54f. Stylet 54 also includes an axial opening 54d. Opening 54d is shaped and dimensioned to receive the sensor assembly 52. In one example, hollow tube 54c is made of stainless steel and sharp tip 54a is hardened, sharpened and welded to the distal end of the hollow tube 54c. The fabrication process of sharp tip 54a slightly magnetizes the tip. Sharp tip 54f is available in many forms and shapes, for instance a trocar tip (as shown in area B in FIG. 2A), a spade tip, or a conical tip.

Sensor assembly 52 includes a sensor tube 52e having an electromagnetic sensor 52f at its distal end 52k and an insulated sensor cable 52a connected to its proximal end 521. The delicate sensor assembly 52 is inserted inside opening 54d of the hollow stylet tube 54c. Electromagnetic sensor 52f must be positioned at a minimum distance form the magnetized tip 54a in order to avoid distortions in the position and orientation readings. Sensor cable 52a includes a twisted pair of insulated wires 52c. The length of cable 52a depends upon its intended use. In one example, cable 52a has a nominal length of 3 meters. The twisted pair of wires 52c is fragile, yet necessarily unconstrained between their exit point at the end of cable 52a and the entrance to the sensor tube 52e. The sensor cable 52a also includes a strength member 52b that extends a short distance from the cable distal end. In one example, strength member 52b is made of Kevlar filament. The cable cross section also features an insulating layer 52g. The sensor assembly 52 also includes a connector 52d at the proximal end of cable 52a. Connector 52d connects the cable 52a to the tracking electronics 32b, as shown in FIG. 1. Connector 52d also contains non-volatile storage circuitry 52h that is used for storing sensor 52f to stylet tip 54a position and orientation offset calibration data. Sensor tube 52e is rigid to compressive force along its axis, but bends like a spline when a point force is applied normal to its axis. In one example, sensor tube 52e is made of Polyamide. As was mentioned above, sensor tube 52e contains the magnetic sensor 52f at its distal end and the twisted pair of wires 52c that electrically connect to the sensor 52f wires. The sensor tube 52e is cut to a precise length per the known length of the stylet cavity 54d and the outer diameter of the sensor tube 52e is less than the inner diameter of the stylet cavity 54d.

Referring to FIG. 2B and FIG. 2C, receiver 54g is made of clear or opaque plastic and has a press-fit design with internal surfaces and form that receive and fit snugly around the distal end of the inserted cable 52a. In some embodiments, receiver 54g includes gripping features and features for connecting to a press-handle (not shown) for high-force insertion medical procedures. Receiver 54g also includes a male friction lock feature 56e, 59e for fixing and stopping the stylet 54 when fully inserted into the cannula 58. The plastic receiver 58b of the outer cannula 58, functions as the female friction lock to the stylet receiver male friction lock features 56e, 59e. The outer diameter of the stylet 54 is less than the inner diameter of the cannula 58 opening 54d. In the embodiment 56 of FIG. 2B, receiver 54g has a central axial opening 54d and the in the embodiment of 59 of FIG. 2C, receiver 54g has an off-axis opening 54d.

FIG. 2D-FIG. 2G, depict four different sensor configuration embodiments including compensated sensor configuration 70, manipulated sensor configuration 90, blind-set shim-rings sensor configuration 110, and blind-set stop-plug sensor configuration 130, respectively. In the compensate sensor configuration 70, the end 52m of sensor 52f is positioned at a distance t from the end 52k of sensor tube 52e. In the manipulated sensor configuration 90, the end 52m of sensor 52f is positioned at a distance t from the end 52k of sensor tube 52e and a sleeve of hot-melt plastic 92 is positioned around the sensor 52f, as shown in FIG. 2E. In the blind-set shim-rings sensor configuration 110, the end 52m of sensor 52f is positioned at a distance t from the end 52k of sensor tube 52e and two heat shrink rings 112a, 112b are positioned separately and coaxially around the two ends 52m, 52n of the sensor 52f, as shown in FIG. 2F. In the blind-set stop-plug sensor configuration 130, the end 52m of sensor 52f is positioned to coincide with the end 52k of sensor tube 52e and a stop-plug 132 is positioned over the ends 52k and 52m, as shown in FIG. 2G. All of these sensor configuration embodiments provide cost reduced apparatuses and methods for positioning and orienting the sensor 52f with respect to the stylet tip 54a and stylet axis 56a, respectively, where the diameter of the sensor tube 52e is significantly smaller than the diameter of the stylet cavity 54b. Cost reduction is realized because one sensor tube 52e outer diameter and length is applicable to a wide range of larger stylet cavity 54b dimensions. This reduction in the sensor assembly product mix enables economy of scale effects in the cost of the sensor assembly models. TABLE 1 shows the attributes associated with each of the configuration options.

TABLE 1
Sensor Configuration Application Matrix

| | Apparatus/Method | | | |
|---|---|---|---|---|
| | | | Blind-set | Blind-set |
| | Compensated | Manipulated | Shim-Rings | Stop-Plug |
| Attribute | 70 | 90 | 110 | 130 |
| Small Batch | X | X | | |
| Large Batch | | | X | X |
| Compensated | X | | | |
| offsets | | | | |
| Corrected | | X | X | X |
| offsets | | | | |
| Offset | 100% | 100% | Sampled | Sampled |
| calibration | | | | |
| Non-volatile | X | Optional | | |
| offset storage | | | | |

Referring to TABLE 1, small batch, low volume, fabrication of the disposable sensor sub-assembly does not apply to precision blind-set components such as shim-rings 112 and stop-plugs 132. Small batches are semi-custom and require the manual means of compensating 70 and/or manipulating 90 the sensor 52f position and orientation with respect to the stylet tip 54a. Conversely, large batch, high volume, fabrication cost-justifies use of the blind-set components. For large batch (high unit count) fabrication, the blind-set components are available, affordable, and stocked in quantity.

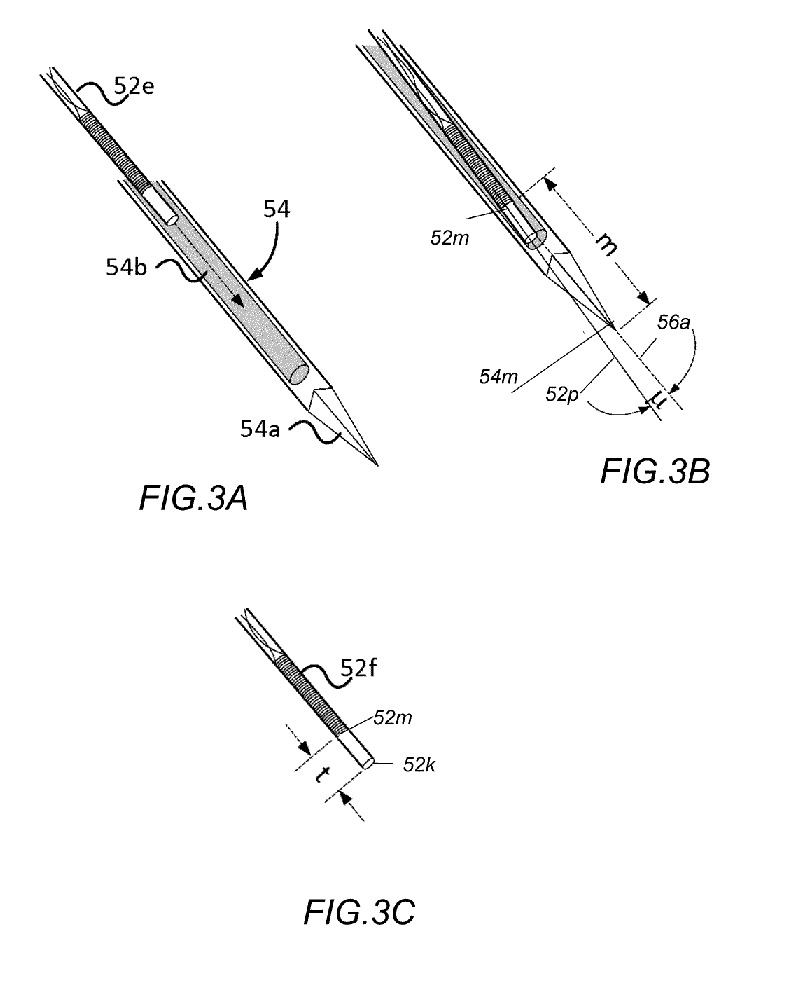

FIG. 3A-FIG. 3C illustrate the offset compensation method for small batch disposable needle fabrication 70. Referring to FIG. 3C, the sensor tube 52e distal end 52k is trimmed to position the sensor end 52m at distance, t, from the sensor tube distal end 52k. The distance, t, is large enough to separate the sensor 52f from the slightly magnetized stylet tip 54a. Referring to FIG. 3A, the sensor tube 52e is inserted fully in the stylet cavity 54b and fastened at the proximal end 54e of the stylet 54. Referring to FIG. 3B, the resulting assembly of the sensor 52f and the stylet 54 is placed in a calibration fixture (not shown) where the sensor position offset, m, and sensor orientation offset, μ are measured and recorded. The sensor position offset m is the distance of the end 52m of sensor 52f from the stylet tip end 54m. The sensor orientation offset μ is the angle between the stylet axis 56a and the sensor tube axis 52p. The sensor position offset m and the sensor orientation offset μ are used as calibration values and are stored in the sensor sub-assembly nonvolatile storage circuitry 52h. The stored calibration values are read by the tracking electronics 32b and applied as compensation during disposable needle tracking.

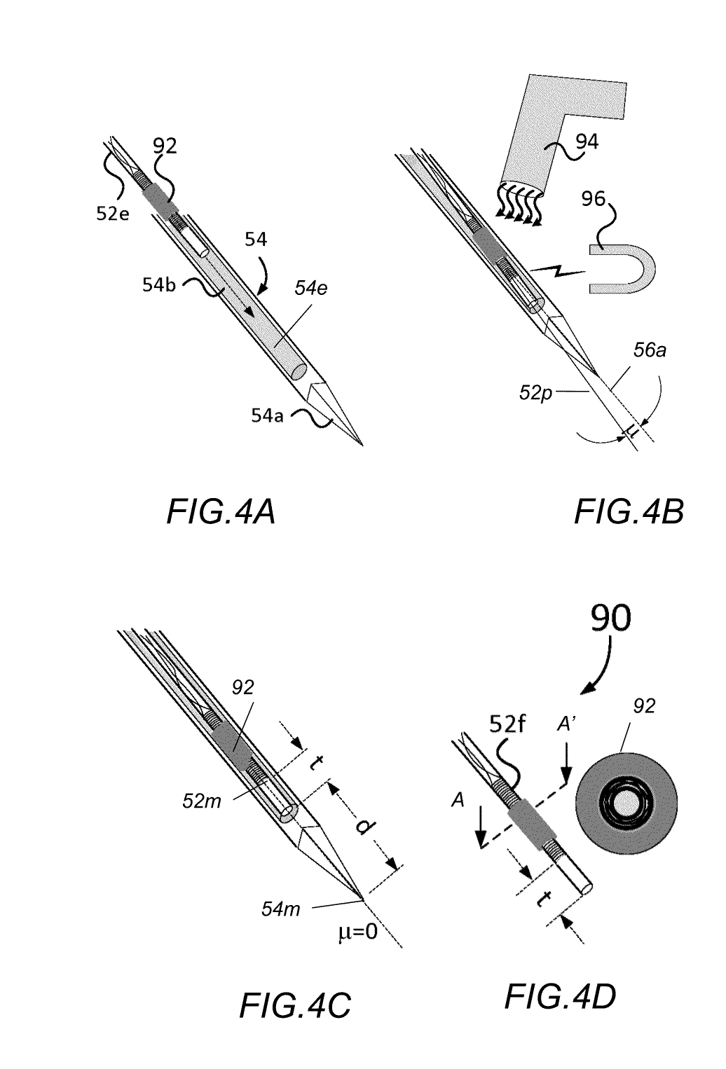

FIG. 4A-FIG. 4D illustrate the sensor manipulation 90 method for small batch disposable needle fabrication. Referring to FIG. 4D, the sensor tube 52e distal end 52k is trimmed to position the sensor end 52m at distance t, from the sensor tube distal end 52k. The distance, t, is large enough to separate the sensor from the slightly magnetized stylet tip 54a. A preformed sleeve of hot-melt plastic 92 is positioned around and tacked to the sensor tube 52e with a heat-gun 94, shown in FIG. 4B. Referring to FIG. 4A, the sensor tube 52e is inserted fully in the stylet cavity 54b and fastened at the proximal end 54e of the stylet 54. Referring to FIG. 4B the resulting assembly of the sensor 52f and the stylet 54 is placed in a calibration fixture (not shown) where the sensor position offset m, and sensor orientation offset μ, calibration values are measured while the stylet tip 54a is heated with a heat-gun 94 to melt and flow the hot-melt sleeve 92. Concurrently, a magnet 96 is introduced to induce a manipulating force on the highly magnetic sensor 52f. The sensor 52f is iteratively manipulated with the magnet 96 until the sensor orientation offset μ is measured to be zero degrees. Referring to FIG. 4C, when the sensor orientation offset μ is set to zero degrees the heat-gun is removed and the hot-melt solidifies to fix the sensor coaxially to the stylet 54. The last iteration calibration position and orientation offset data, although very close to m=t+d and μ=zero degrees, respectively, can optionally be written to the sensor nonvolatile storage circuitry 52h. This small batch manipulation method 90, corrects the sensor offsets, and is preferable to the small batch compensation method 70 if the end-user requires the sensor to be physically coaxial with the stylet.

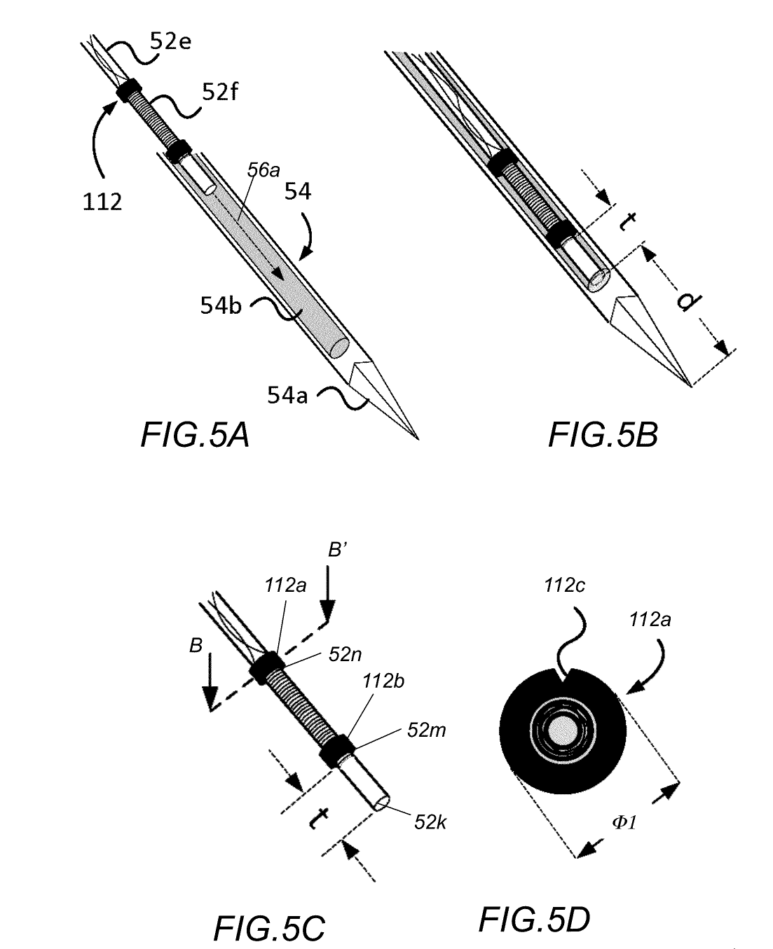

FIG. 5A-FIG. 5D illustrate the sensor blind-set method 110 used for large batch disposable needle fabrication. Referring to FIG. 5C, the sensor tube 52e distal end 52k is trimmed to position the sensor end 52m at a distance, t, from the sensor tube distal end 52k. The distance, t, is large enough to separate the sensor 52f from the slightly magnetized stylet tip 54a. At least two preformed heat shrink rings 112a, 112b are positioned separately and coaxially on the distal end of the sensor tube 52e so that they around the ends 52m, 52n of the sensor 52f. The rings 112a, 112b are heated using conventional means, such as a hole-in-hot-block fixture, and collapsed to a tight fit around the sensor tube 52e. The heating time and temperature are precisely controlled, to achieve a predetermined outer diameter, φ1, of the heat shrink ring 112a, 112b. The outer diameter, φ1, is slightly smaller than the diameter of the intended stylet cavity 54b. Each heat shrink ring 112a, 112b is scored 112c parallel to its axis, to enable pass-by of trapped air as the sensor tube 52e is inserted into the stylet cavity. Referring to FIG. 5A, the sensor tube 52e is inserted into the stylet cavity 54a to maximum depth. The maximum depth is confirmed by visual inspection of the position of the proximal end 521 of the sensor tube relative to the proximal end 54e of the stylet cavity. The sensor tube 52e is fastened by means described herein at the proximal end of stylet 54. Referring to FIG. 5B, the sensor 54f is now fixed at a known distance, m=t+d, from the stylet tip end 54m and is coaxially oriented to the stylet axis. A random sample subset of each batch of disposable needle assemblies fabricated with this method 110 may be measured to ensure the expected position and orientation offsets are in fact m=t+d and μ=zero degrees, respectively. With this blind-set method 110, there is no need to store calibration offset data in the nonvolatile storage circuitry 52h because the resulting offset data are known and invariable for each stylet model.

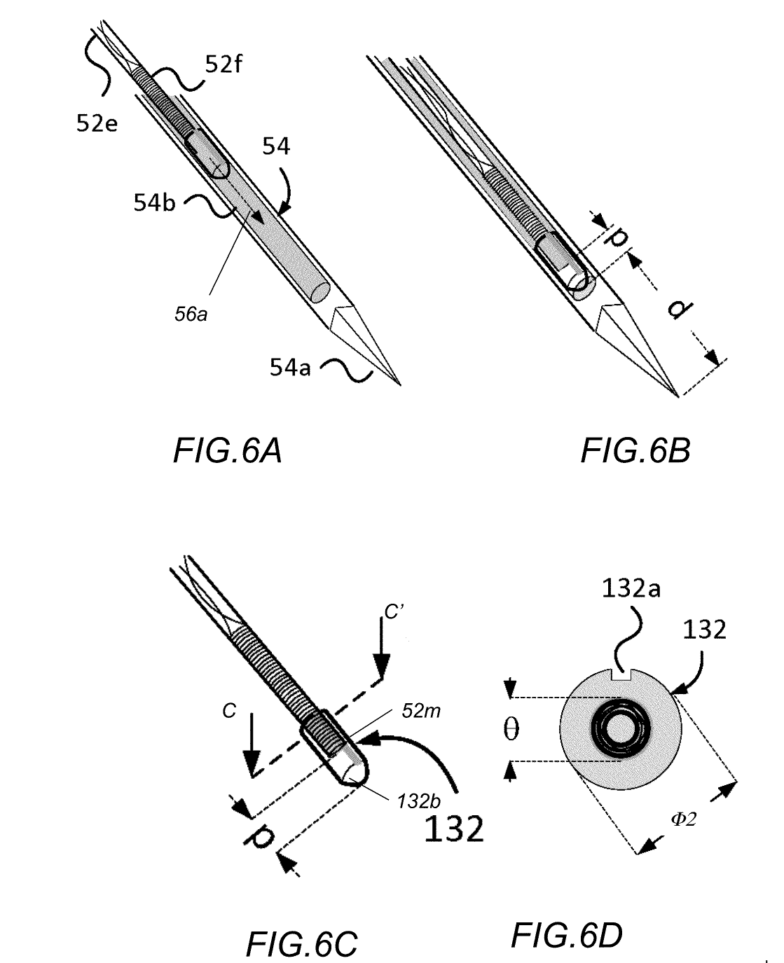

FIG. 6A-FIG. 6D, illustrates the sensor blind-set method 130 used for large batch disposable needle fabrication. Referring to FIG. 6C, the sensor tube 52e distal end 52k is trimmed to coincide with the distal end 52m of the sensor 52f and then a stop-plug 132 is placed over it. Stop-plug 132 is selected so that the outer diameter, 12, is slightly smaller than the inner diameter of the stylet cavity 54b, and the inner diameter, θ, is slightly larger than the outer diameter of sensor tube 52e. The stop-plug 132 receives the distal end 52k of the sensor tube 52e to a maximum stop depth and thereby the sensor 52f tip 52m is placed at a known distance, p, from the distal end 132b of the stop-plug 132. The distance, p, is large enough to separate the sensor from the slightly magnetized stylet tip 54a. Optionally, a small amount of quick set-adhesive is applied to fix the sensor tip 52m in the stop-plug 132. The stop-plug 132 features a groove 132a parallel to its axis, to enable pass-by of trapped air as the sensor tube 52e is inserted into the stylet cavity. Referring to FIG. 6A the sensor tube 52e is inserted into the stylet cavity 54b to maximum depth. The maximum depth is confirmed by visual inspection of the position of the proximal end 521 of the sensor tube 52e relative to the proximal end 54e of the stylet cavity. The sensor tube is fastened by means described herein at the proximal end of stylet 54. Referring to FIG. 6B, the sensor 54f is now fixed at a known distance, n=p+d, from the stylet tip 54a and is coaxially oriented to the stylet axis 56a. A random sample subset of each batch of disposable needle assemblies fabricated with this method 130 may be measured to ensure the expected position and orientation offsets are in fact n=p+d and μ=zero degrees, respectively. With this blind-set method 130, there is no need to store calibration offset data in the nonvolatile storage circuitry 52h because the resulting offset data are known and invariable for each stylet model.

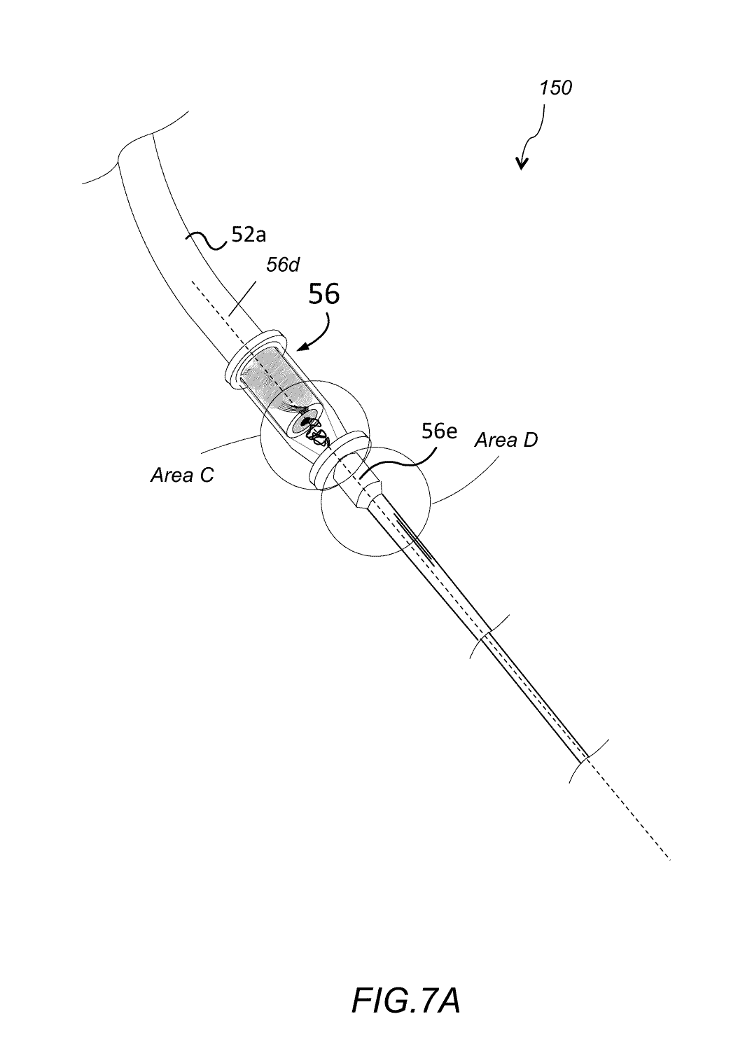

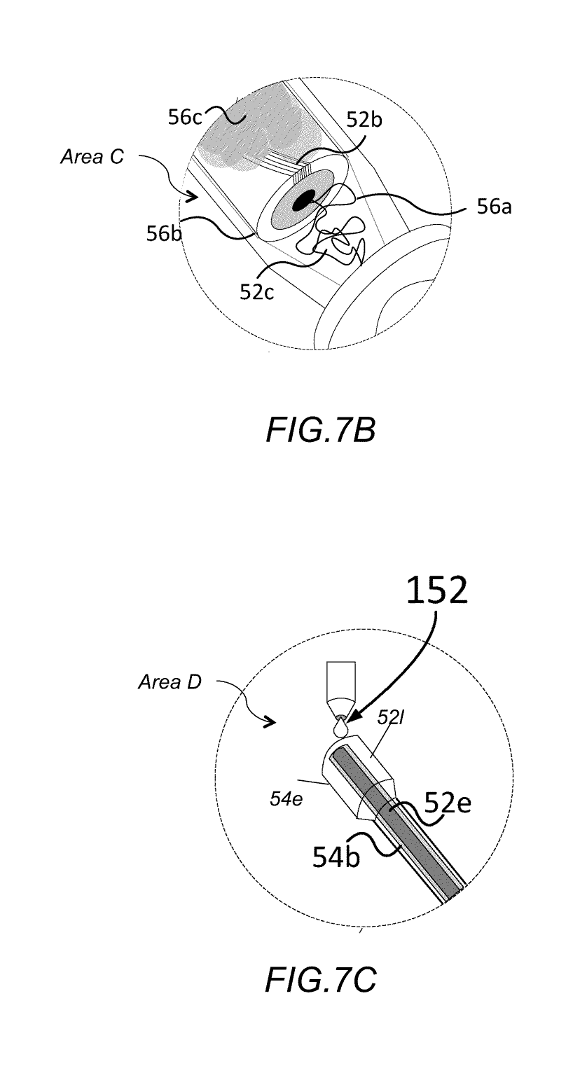

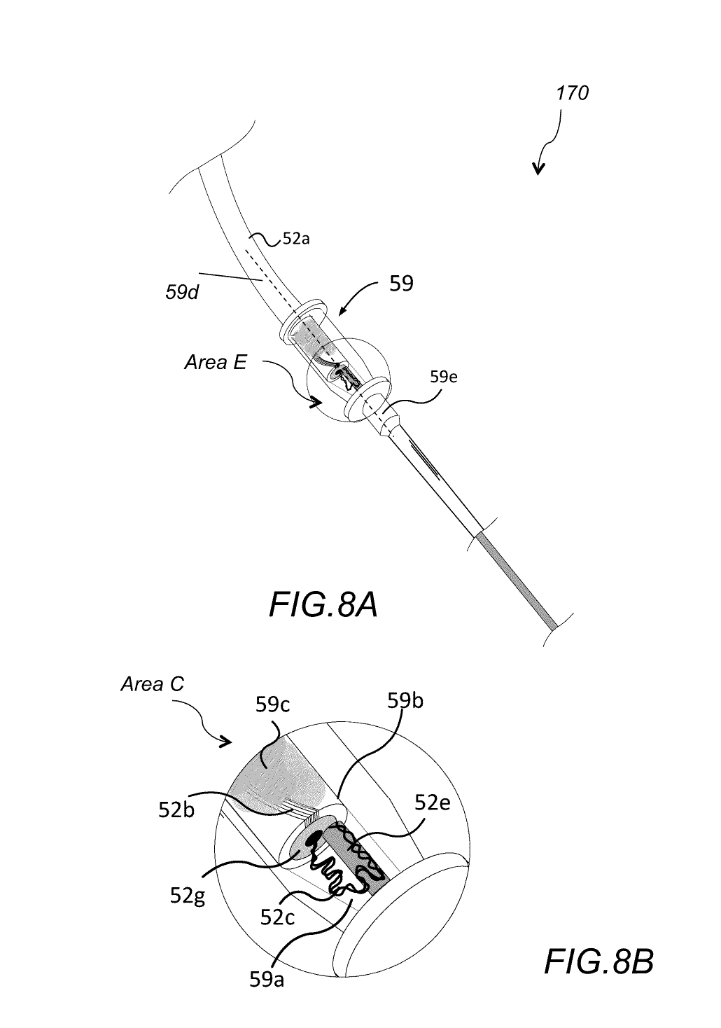

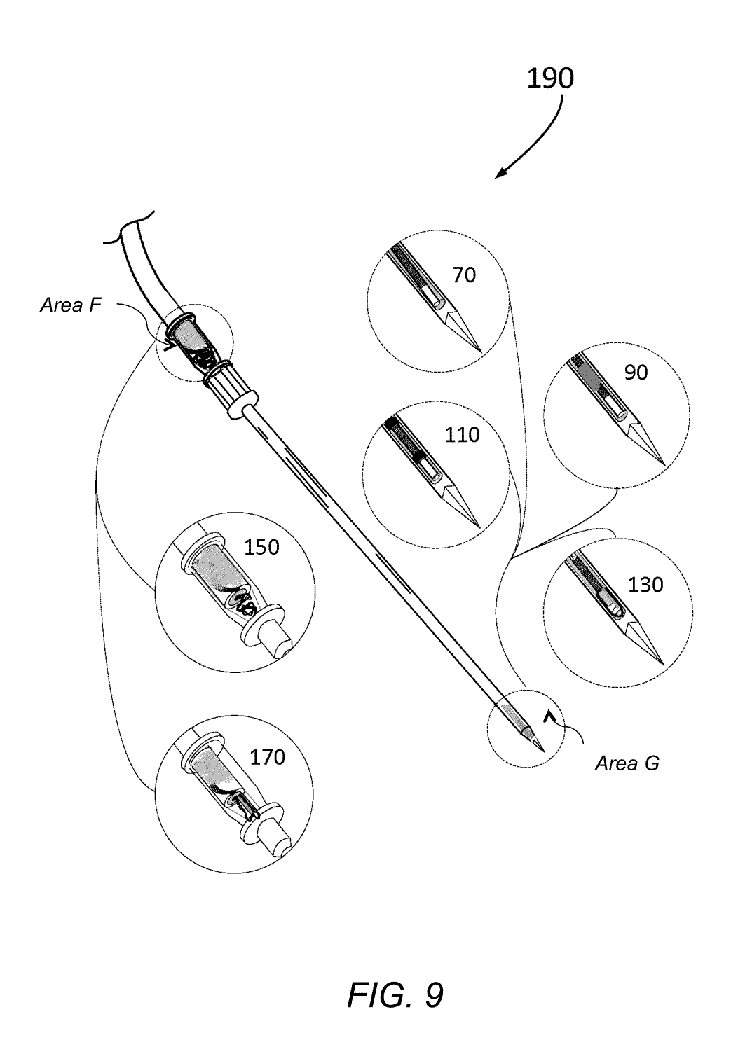

FIG. 7A and FIG. 8A depict two stylet receiver configuration apparatuses and methods 150, 170, respectively, for securing and positioning the sensor assembly cable 52a in the receiver 56. The receiver cavity axis 56d in configuration 150 is coaxial to the stylet cavity 54b, as shown in FIG. 7A. The receiver cavity axis 59d in configuration 170 is parallel to but offset from the stylet cavity axis, as shown in FIG. 8A.

Referring to FIG. 7C, the sensor tube 52e proximal end 521 is fixed in the proximal end 54e of stylet cavity 54b with a droplet of quick-set adhesive 152. Referring to FIG. 7B, the receiver cavity 56b features a slight taper to act as a press-fit to the sensor cable 52a. The receiver cavity 56b features a hard stop for the inserted cable end, and the hard stop allows a remaining cavity volume 56a wherein the unconstrained twisted pair 52c is enclosed. Adhesive 56c is applied to the press-fit surface of the receiver cavity 56b. The sensor cable strength member 52b is folded back along the sensor cable 52a. The sensor cable is inserted into the press-fit receiver cavity to maximum depth, with care given to ensure the sensor twisted pair 52c is loosely housed in the remaining cavity volume 56a. Examples of quick-set adhesive material applied in this configuration option 150 are hot-melt preform and/or Cyanoacrylate.

Referring to FIG. 8A, configuration 170 features the press-fit receiver cavity axis 59d being offset from the stylet 54 axis. The axis offset is such that the sensor cable insulating material 52g is aligned to contact and lightly compress against the extended proximal end of the sensor tube 52e. The receiver cavity 59b features a slight taper to act as a press-fit to the sensor cable 52a, as shown in FIG. 8B. The receiver cavity 59b features a hard stop for the inserted cable end, and the hard stop allows a remaining cavity volume 59a wherein the unconstrained twisted pair 52c is enclosed. Adhesive 59c is applied to the press-fit surface of the receiver cavity 59b. The sensor cable strength member 52b is folded back along the sensor cable 52a. The sensor cable is inserted into the press-fit receiver cavity to maximum depth. Sensor tube 52e has been pre-cut to a specified length such that the proximal end 521 of the sensor tube contacts the cable insulating material 52g, thus lightly compressing the proximal end of the sensor tube 52e. Attention is given to ensure the sensor twisted pair 52c is loosely housed in the remaining cavity volume 56a. The compression means eliminates the need to glue the proximal end of the sensor tube to the proximal end of the stylet cavity 54b, thereby further reducing the labor component of the disposable needle 190 cost.

Referring to FIG. 9, the sensor-loaded stylet 54 is inserted into the outer cannula 58 with sufficient force to mate the stylet receiver male feature 56e with the female cannula receiver 58b. The magnified views 150, 170, 70, 90, 110 and 130 illustrate the permutations of cost reducing apparatuses and methods described herein. The stylet tip 54a is oriented as designed relative to the cannula tip 58a, and the sensor 52f is precisely positioned at a known distance and orientation relative to the stylet tip and thus the cannula tip.

Several embodiments of the present invention have been described. Nevertheless, it will be understood that various modifications may be made without departing from the spirit and scope of the invention. Accordingly, other embodiments are within the scope of the following claims.

What is claimed is:

1. An electromagnetic needle tracking system comprising:
a needle assembly comprising a needle stylet and a sensor assembly; wherein said needle stylet comprises an elongated hollow tube comprising an open proximal end and a distal end comprising a needle tip; and wherein said sensor assembly comprises an elongated body, and a sensor attached to the elongated body and wherein said elongated body is shaped and dimensioned to be inserted into said elongated hollow tube, and wherein said sensor is configured to measure position and angular orientation data when placed within an electromagnetic field;
a calibration system comprising a calibration fixture and wherein said calibration system is configured to measure the sensor's position and angular orientation for a known needle tip position and angular orientation within the calibration fixture and to calculate a position offset and an angular orientation offset of the sensor relative to the needle tip position and angular orientation; and
a computing system for computing position and angular orientation data of the needle tip by adding the sensor position offset and angular orientation offset to the measured position and angular orientation data, respectively.

2. The system of claim 1, further comprising a non-volatile storage circuitry configured to store the calculated sensor position and angular orientation offsets.

3. A needle assembly comprising:
a needle stylet comprising an elongated hollow tube and a needle and wherein the elongated hollow tube extends along a first axis and comprises an open proximal end and a closed distal end, and wherein the needle is attached to the closed distal end of the elongated hollow tube and comprises a tip end that extends a first distance from the closed distal end of the elongated hollow tube along the first axis;
a sensor assembly comprising an elongated body extending along a second axis, and a sensor placed at a second distance from the distal end of said elongated body and wherein said elongated body is shaped and dimensioned to be inserted into said elongated hollow tube so that the distal end of the elongated body is placed in contact with the closed distal end of the elongated hollow tube, and wherein said sensor is configured to measure position and angular orientation data when placed within an electromagnetic field; and
a computing system for computing the position and angular orientation of the tip end of the needle by adding the sum of the first and the second distances to the measured position data and by adding the angular difference between the first and second axes to the measured angular orientation data, respectively.

4. A needle assembly comprising:
a needle stylet comprising an elongated hollow tube and a needle and wherein the elongated hollow tube extends along a first axis and comprises an open proximal end and a closed distal end, and wherein the needle is attached to the closed distal end of the elongated hollow tube and comprises a tip end that extends a first distance from the closed distal end of the elongated hollow tube along the first axis;
a sensor assembly comprising an elongated body extending along a second axis, and a sensor located at a second distance from the distal end of said elongated body and wherein said elongated body is shaped and dimensioned to be inserted into said elongated hollow tube so that the distal end of the elongated body is placed in contact with the closed distal end of the elongated hollow tube, and wherein said sensor is configured to measure position and angular orientation data when placed within an electromagnetic field;
means for fixing the sensor's angular orientation within said elongated hollow tube to be coaxial with said first axis; and
a computing system for computing the position of the tip end of the needle by adding the sum of the first and the second distances to the measured position data.

5. The assembly of claim 4, wherein said means for fixing the sensor's angular orientation comprises a sleeve of hot-melt plastic and wherein said sleeve is configured to be positioned around the elongated body and to be tacked to the elongated body by heating.

6. The assembly of claim 5, wherein the sensor's angular orientation is fixed to be coaxial with said first axis by iteratively heating and melting the sleeve of hot-melt plastic, orienting the elongated body, cooling and solidifying the sleeve of hot-melt plastic and measuring the resulting angular difference between the first and second axes until the elongated body is coaxial with the elongated hollow tube.

7. The assembly of claim 6, wherein said sensor comprises a magnetic sensor and said elongated body is oriented within the elongated hollow tube by applying a magnetic force.

8. The assembly of claim 4, wherein said means for fixing the sensor's angular orientation comprises first and second heat-shrink rings, wherein said first and second heat-shrink rings are positioned coaxially and around the sensor's first and second ends, respectively, and subsequently said sensor assembly is inserted into said elongated hollow tube and said heat-shrink rings are heated at a controlled temperature and for a controlled time period until the outer diameter of the heat-shrink rings expands to be slightly smaller than the inner diameter of the elongated hollow tube, and thereby orienting and fixing the elongated body coaxially with the elongate hollow tube.

9. The assembly of claim 8, wherein the outer surface of each of said first and second heat-shrink rings comprises a groove and said groove is oriented parallel to the ring's axis.

10. A needle assembly comprising:
a needle stylet comprising an elongated hollow tube and a needle and wherein the elongated hollow tube extends along a first axis and comprises an open proximal end and a closed distal end, and wherein the needle is attached to the closed distal end of the elongated hollow tube and comprises a tip end that extends a first distance from the closed distal end of the elongated hollow tube along the first axis;
a sensor assembly comprising an elongated body, a sensor located at the distal end of said elongated body and a stop-plug configured to be placed over the elongated body's distal end and wherein said elongated body is shaped and dimensioned to be inserted into said elongated hollow tube so that distal end of the stop-plug is in contact with the closed distal end of the elongated hollow tube and wherein said stop-plug comprises an outer diameter slightly smaller than the inner diameter of the elongated hollow tube and is configured to orient the elongated body coaxially with the elongated hollow tube and wherein the stop-plug comprises an inner diameter slightly larger than the outer diameter of the elongated body and is configured to receive and place the elongated body's distal end at a second distance from the stop-plug's distal end; and
a computing system for computing the position of the tip end of the needle by adding the sum of the first and the second distances to the measured position data.

11. The assembly of claim 10, wherein the outer surface of the stop-plug comprises a groove and said groove is oriented parallel to the stop-plug's axis.

12. The assembly of claim 1, wherein the needle stylet further comprises a stylet receiver attached to the proximal end of the elongated hollow tube and wherein the needle assembly further comprises means for attaching the elongated body's proximal end to the stylet receiver.

13. The assembly of claim 12, wherein the elongated body's proximal end is attached to the stylet receiver with an adhesive.

14. The assembly of claim 13, wherein said adhesive comprises one of cyanoacrylate, epoxy, hot melt or solvent bonding.

15. The assembly of claim 12, wherein the stylet receiver comprises a cavity and said cavity is tapered.

16. The assembly of claim 15, wherein the sensor assembly further comprises an insulated cable and a pair of twisted insulated wires connected to the distal end of the insulated cable and wherein the distal end of the insulated cable is inserted in the receiver cavity and the proximal end is connected to the computing system and wherein the tapered cavity provides a hard stop for the inserted distal end of the insulated cable.

17. The assembly of claim 12, wherein the stylet receiver comprises a cavity extending coaxially with the elongated hollow tube.

18. The assembly of claim 12, wherein the stylet receiver comprises a cavity extending parallel to but offset from the elongated hollow tube.

19. The assembly of claim 3 further comprising a non-volatile storage circuitry configured to store calibration data comprising the first and second distances, the sum of the first and second distances, and the angular difference between the first and second axes.

20. The assembly of claim 1 further comprising an outer cannula and wherein said needle stylet is configured to be inserted into said outer cannula.
